(19) United States
(12) Patent Application Publication
(10) Pub. No.: US 2010/0168718 A1
(43) Pub. Date: Jul. 1, 2010

(54) Extension Tube Assembly

(75) Inventors: Marc Bellisario, Tewksbury, MA (US); Mark Lotito, Wrentham, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/636,093

(22) Filed: Dec. 11, 2009

Related U.S. Application Data
(60) Provisional application No. 61/141,518, filed on Dec. 30, 2008.

(51) Int. Cl. A61M 25/18

(52) U.S. Cl. 604/533

(57) ABSTRACT

An extension tube assembly for fluidly connecting a catheter assembly to a hemodialysis machine includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens. The coupling mechanism includes a first coupling mounted on a distal end of the first extension segment and a second coupling mounted on a proximal end of the second extension segment. The first and second couplings cooperate to provide a releasable connection therebetween to permit selective connection and disconnection of the first and second extension segments.

This application claims priority from U.S. provisional application Ser. No. 61/141,518 filed Dec. 30, 2008, the entirety of which is incorporated herein by reference.

### BACKGROUND

1. Technical Field

The present disclosure generally relates to catheter assemblies. More particularly, the present disclosure relates to extension tube assemblies for fluidly coupling a catheter assembly to a hemodialysis machine.

2. Description of the Related Art

Catheters are flexible medical instruments which facilitate the withdrawal and introduction of fluids from and to body cavities, ducts, and vessels. Catheter instrumentation may have particular application in a hemodialysis procedure where blood is withdrawn from a blood vessel for treatment, and subsequently returned to the blood vessel for circulation. Known hemodialysis catheter assemblies include multiple lumen catheters, such as dual lumen or triple-lumen catheters, which permit bi-directional fluid flow within the catheter whereby one lumen is dedicated for withdrawal of blood from a vessel and the other lumen is dedicated for return of treated blood to the vessel. During an exemplary hemodialysis procedure, a multiple lumen catheter is inserted into a body and blood is withdrawn through an arterial lumen of the catheter. The removed blood is directed to a hemodialysis machine, via extension tubes, which dialyzes, or purifies, the blood to remove waste and toxins. The dialyzed blood is returned to the patient through a venous lumen of the catheter.

Extension tubes which may be glued or molded onto the catheter hub, carry blood from and into the lumens of the catheter during the hemodialysis procedure. Usually, a distal end of the extension tube is attached to the catheter hub and a proximal end of the extension tube includes a luer adapter fixed thereto. The luer adapter facilitates fluid interconnection between the hemodialysis machine and the extension tubes. Between hemodialysis procedures, a clinician caps and clamps the extension tubes, to minimize the possibility of blood leakage and reduce the risk of infection by microorganisms entering the bloodstream.

### SUMMARY

The present disclosure relates to an extension tube assembly for fluidly connecting a catheter assembly to a hemodialysis machine. The extension tube assembly includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens and includes a first coupling and a second coupling. The first coupling is mounted on a distal end of the first extension segment. The first coupling includes an elongate member extending distally therefrom. The elongate member having a barb disposed around a distal end thereof. The second coupling is mounted on a proximal end of the second extension segment. The second coupling includes a recess adapted to securely receive the elongate member to establish a secure connection between the first and second couplings. The first and second couplings cooperate to provide a releasable connection therebetween to permit selective connection and disconnection of the first and second extension segments. The first and second couplings collectively form a bore when connected to each other, the bore being configured to establish fluid communication between the first and second lumens.

In one embodiment, the extension tube assembly further includes an end cap mountable on the first coupling of the first extension segment when the second coupling of the second extension segment is released from the first coupling of the first extension segment. The end cap is dimensioned to substantially close the first lumen of the first extension segment to prevent passage of fluid.

In one embodiment, the first extension segment includes a luer adapter mounted on a proximal end thereof.

The present disclosure relates to an extension tube assembly for fluidly connecting a catheter assembly to a hemodialysis machine. The extension tube assembly includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens and includes a first coupling and a second coupling. The first coupling is mounted on a distal end of the first extension segment. The first coupling includes an elongate member extending distally therefrom. The second coupling is mounted on a proximal end of the second extension segment. The second coupling includes a recess adapted to receive the elongate member. The first and second couplings jointly form a bayonet coupling mechanism to provide a releasable connection therebetween to permit selective connection and disconnection of the first and second extension segments. The coupling mechanism includes a pin and a slot. The pin protrudes radially from the elongate member. The slot is formed on the second coupling. The slot has a curved profile.

In one embodiment, the second extension segment includes a luer adapter for fluid connection to the hemodialysis machine.

The present disclosure relates to an extension tube assembly for fluidly connecting a catheter assembly to a hemodialysis machine. The extension tube assembly includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens and includes a first coupling and a second coupling. The first coupling is mounted on a distal end of the first extension segment. The first coupling includes at least one flexible locking extension extending distally therefrom. The locking extension has an opening formed thereon. The second coupling is mounted on a proximal end of the second extension segment. The second coupling includes at least one lug extending radially therefrom. The lug is adapted to be received in the opening of the at least one flexible locking extension to establish a secure connection between the first and second couplings. The first and second couplings cooperate to provide a releasable connection therebetween to permit selective connection and disconnection of the first and second extension segments. The first and second couplings collectively form a bore when connected to each other. The bore is configured to establish fluid communication between the first and second lumens.

In one embodiment, the second extension segment includes a luer adapter for connection to the hemodialysis machine.

The present disclosure relates to an extension tube assembly for fluidly connecting a catheter assembly to a hemodialysis machine. The extension tube assembly includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens and includes a first coupling and a second coupling. The first coupling is mounted on a distal end of the first extension segment. The first coupling includes an extension extending distally therefrom. The extension has an external thread formed thereon. The second coupling is mounted on a proximal end of the second extension segment. The second coupling includes a recess. The recess has an internal thread formed therein. The internal thread is adapted to threadly engage the external thread to establish a secure connection between the first and second couplings. The first and second couplings cooperate to provide a releasable connection therebetween to permit selective connection and disconnection of the first and second extension segments. The first and second couplings collectively form a bore when connected to each other, the bore being configured to establish fluid communication between the first and second lumens.

In one embodiment, the second extension segment includes a luer adapter for fluid connection to the hemodialysis machine.

The present disclosure relates to an extension tube assembly for fluidly connecting a catheter assembly to a hemodialysis machine. The extension tube assembly includes an extension tube member and a coupling mechanism. The extension tube member includes first and second extension segments. The first extension segment is adapted for connection to a catheter hub and defining a first lumen. The second extension segment is adapted for fluid connection to a hemodialysis machine and defining a second lumen. The coupling mechanism is adapted to connect the first and second extension segments to establish fluid communication between the first and second lumens and includes a first coupling and a second coupling. The first coupling is mounted on a distal end of the first extension segment. The first coupling including a body and first and second arms pivotally secured to the body. Each of the first and second arms having a locking detent at a distal end thereof. The second coupling is mounted on a proximal end of the second extension segment, the second coupling including a first and second locking notches formed thereon. Each notch is adapted to securely receive at least one of the locking detents to establish a secure connection between the first and second couplings. The first and second couplings cooperate to provide a releasable connection therebetween to permit selective connection and disconnection of the first and second extension segments. The first and second couplings collectively form a bore when connected to each other, the bore being configured to establish fluid communication between the first and second lumens.

In one embodiment, the second extension segment includes a luer adapter for fluid connection to the hemodialysis machine.

### BRIEF DESCRIPTION OF THE DRAWINGS

Various embodiments of the presently disclosed extension tube assemblies are described herein with references to the accompanying drawings, wherein:

FIG. 1 is a side view of a medical system fluidly connected to a hemodialysis machine;

FIG. 2 is a side cross-sectional view of the medical system shown in FIG. 1;

FIG. 3 is a perspective view of a portion of the medical system shown in FIG. 1 with end caps mounted on the first extension tubes of the extension tube assembly;

FIG. 4 is a perspective view of a portion of the medical system shown in FIG. 1 with an end cap mounted on a first extension tube of the extension tube assembly;

FIG. 5 is an exploded rear view of a portion of the medical system shown in FIG. 1; and

FIG. 6 is an exploded front view of a portion of the medical system shown in FIG. 1;

FIG. 7 is a side cross-sectional view of a coupling mechanism of the extension tube assembly of the medical system shown in FIG. 1;

FIG. 8 is a perspective view of another embodiment of a medical system with bayonet coupling mechanisms;

FIG. 9 is a perspective exploded view of the medical system shown in FIG. 8;

FIG. 10 is a side cross-sectional view of the bayonet coupling mechanism of the medical system shown in FIG. 8;

FIG. 11 is a perspective view of another embodiment of a medical system with snap fit mechanisms;

FIG. 12 is a side exploded view of the medical system shown in FIG. 11;

FIG. 13 is a side cross-sectional view of the snap fit mechanism shown in FIG. 12;

FIG. 14 is a perspective view of another embodiment of a medical system with threaded mechanisms;

FIG. 15 is side cross-sectional view of the threaded mechanism of the medical system shown in FIG. 14;

FIG. 16 is a perspective view of another embodiment of a medical system with locking detent mechanisms;

FIG. 17 is a side exploded view of the medical system shown in FIG. 16; and

FIG. 18 is a side cross-sectional view of the locking detent mechanism of the medical system shown in FIG. 16.

### DETAILED DESCRIPTION OF THE EMBODIMENTS

In the discussion that follows, the term “proximal” or “trailing” will refer to the portion of a structure that is closer to a clinician, while the term “distal” or “leading” will refer to the portion of a structure that is farther from the clinician. As used herein, the term “subject” refers to a human patient or other animal. The term “clinician” refers to a doctor, nurse or other care provider and may include support personnel.

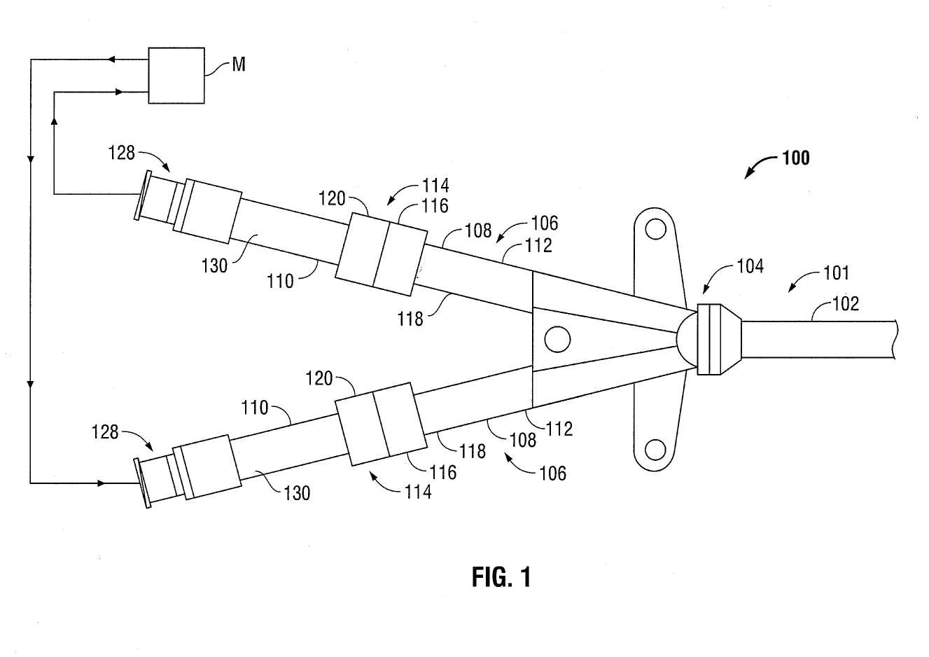

FIG. 1 shows a medical system 100 for use during a hemodialysis procedure. The medical system 100 is fluidly connected to a hemodialysis machine M which dialyzes blood extracted from a subject through medical system 100 and returns dialyzed blood to the subject through medical system 100. Medical system 100 generally includes a catheter assembly 101, extension tube assemblies 106, and a coupling mechanism 114. Catheter assembly 101 includes a catheter hub 104 and an elongated catheter 102 extending distally from catheter hub 104.

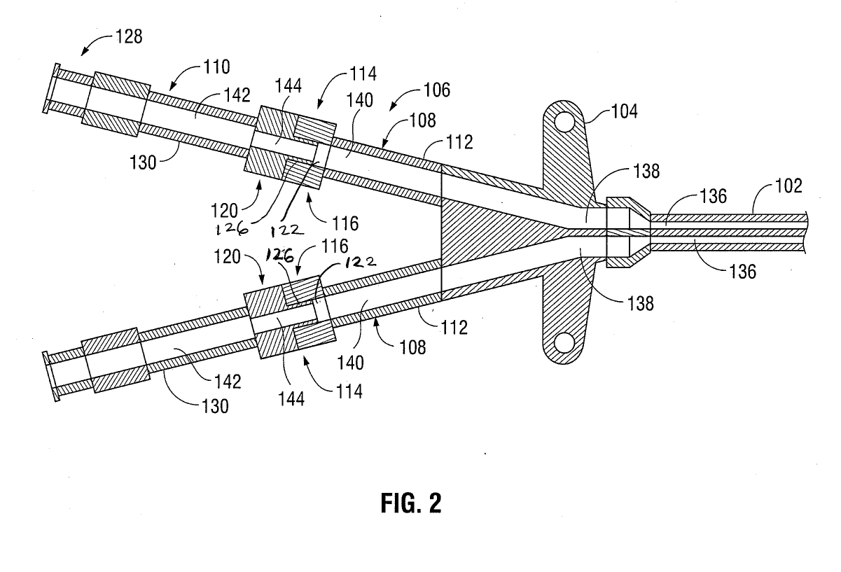

As seen in FIG. 2, the elongated catheter 102 defines at least one fluid channel 136 extending therethrough. In the embodiment depicted in FIG. 2, elongated catheter 102 has two channels 136 arranged in parallel with respect to each other. Each fluid channel 136 is fluidly connected to catheter hub 104. Catheter hub 104 defines at least one conduit 138 adapted to transport fluids therethrough. In one embodiment, catheter hub 104 incorporates two conduits 138. Each conduit 138 is fluidly coupled to fluid channel 136 of elongated catheter 102. Catheter hub 104 is in turn fluidly coupled to extension tube assemblies 106.

Extension tubes assemblies 106 serve as conduits for transferring fluid between hemodialysis machine M and catheter hub 104. However, those skilled in the art will recognize that extension tube assemblies 106 may be employed in other medical and non-medical applications. Each extension tube assembly 106 includes first and second extension segments 108, 110. First extension segment 108 defines a first lumen 140 and second extension segment 110 defines a second lumen 142. As shown in FIGS. 1 and 2, first extension segment 108 is adapted for connection to catheter hub 104, whereas second extension segment 110 is adapted for fluid connection to hemodialysis machine M. Specifically, the distal ends 112 of first extension segments 108 are attached to catheter hub 104 with any suitable fastening means. For example, distal end 112 may be molded onto catheter hub 104, or adhesives may bond distal end 112 to catheter hub 104.

Medical system 100 further includes a coupling mechanism 114 adapted to connect first and second extension segments 108, 110 together to establish fluid communication between first and second lumens 140, 142. Coupling mechanism 114 includes first and second couplings 116, 120 which are associated with the first and second extension segments 108, 110, respectively. First and second couplings 116, 120 cooperate to provide a releasable connection therebetween to thereby permit selective connection and disconnection of first and second extension segments 108, 110. In this embodiment, second coupling 120 frictionally engages first coupling 116 to connect first extension segment 108 to second extension segment. More specifically, first coupling 116 defines a bore 122 (See FIG. 2) dimensioned to receive an elongate member 126 (See FIG. 2) protruding from second coupling 120. The outer diameter of elongate member 126 is substantially equal to (or slightly larger than) the inner diameter of bore 122. As a consequence, elongate member 126 of second coupling 120 is configured to enter bore 122 to establish a frictional connection between first and second couplings 116, 120. In particular, first coupling 116 is attached to the proximal end 118 of first extension segment 108 and second coupling 120 is secured to the distal end 124 of second extension segment 110. First and second couplings 116, 120 collectively form a bore 144 configured to establish fluid communication between first and second lumens 140, 142 of first and second extension segments 108, 110. In certain embodiments, either first or second couplings 116, 120 includes a check valve (not shown) for regulating fluid flow.

Medical system 100 further includes a luer adapter 128 secured to each of the proximal ends 130 of second extension segments 110. Each luer adapter 128 is configured for fluid connection to hemodialysis machine M. Although the drawings show luer adapters 128, it is contemplated that medical system 100 may include any other suitable connection devices capable of establishing fluid communication between hemodialysis machine M and extension tube assemblies 106.

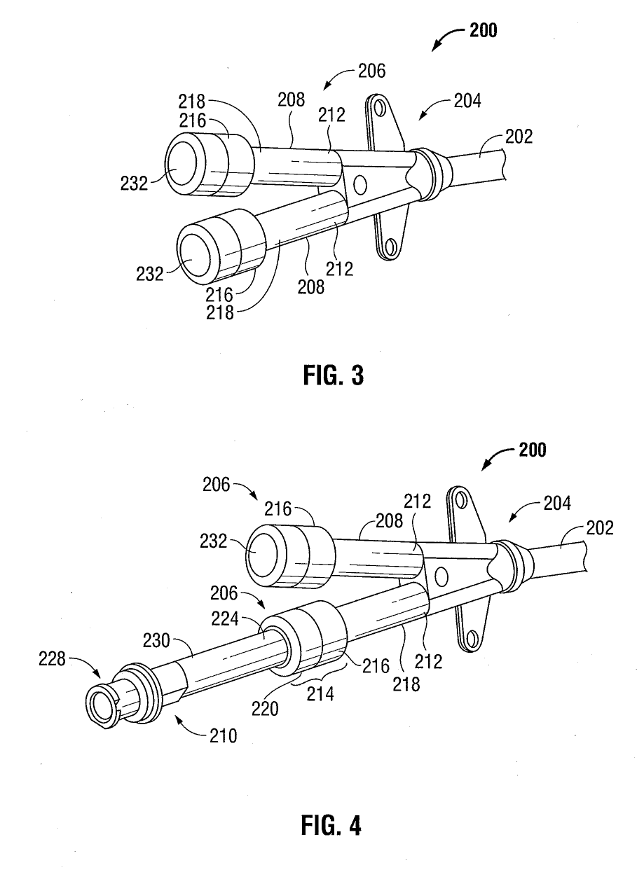

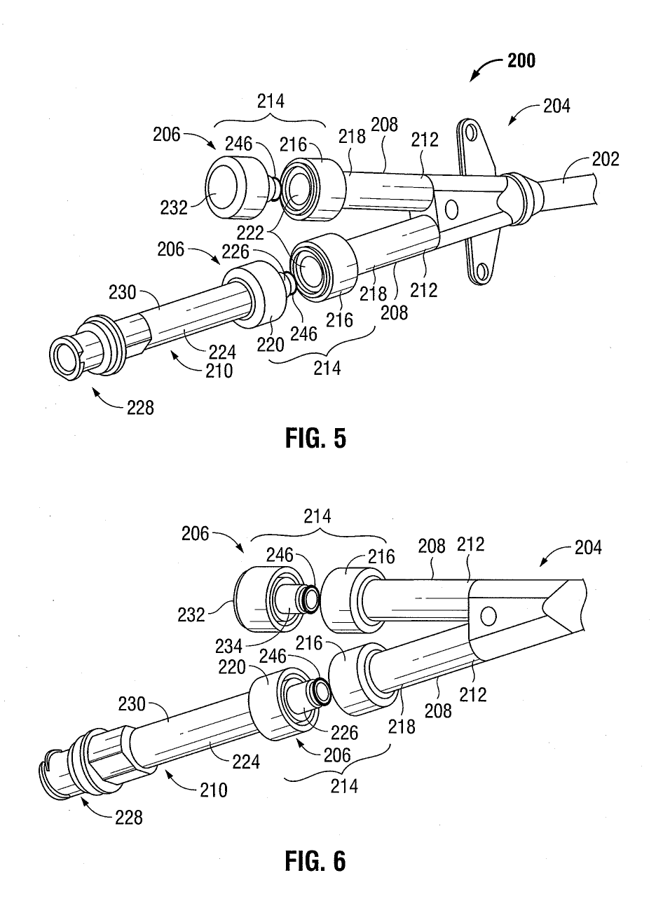

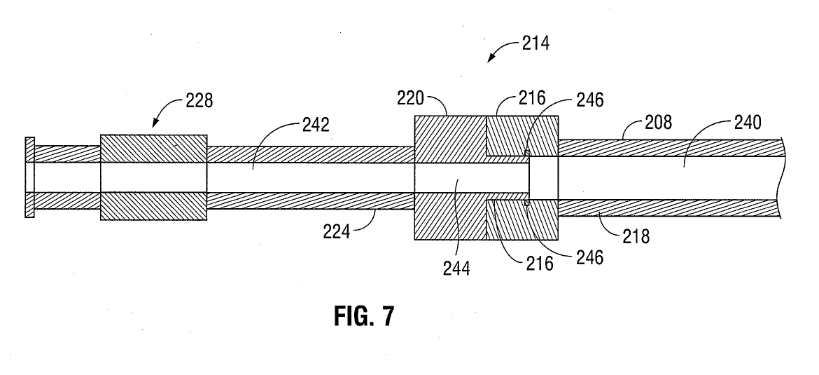

FIGS. 3-7 illustrate an alternative embodiment of medical system 200 which includes a coupling mechanism 214. Medical system 200 is substantially similar to medical system 100 except for coupling mechanism 214. Coupling mechanism 214 includes an interference fit mechanism which is adapted to connect first and second extension segments 208, 210 to establish fluid communication between first and second lumens 240, 242. Coupling mechanism 214 includes first and second interference couplings 216, 220. First and second interference couplings 216, 220 collectively provide a releasable connection therebetween to thereby allow selective connection and disconnection of first and second extension segments 208, 210. First interference coupling 216 is attached to proximal end 218 of first extension segment 208 and second interference coupling 220 is fixed to the distal end 224 of second extension segment 210. As shown in FIGS. 5-7, first interference coupling 216 defines a locking recess 222 and second interference coupling 220 includes an elongated member 226 with a locking barb 246 at a distal end thereof. Although the drawings show first interference coupling 216 with locking recess 222 and second interference coupling 220 with a locking barb 246, locking recess 222 may be located in second interference coupling 220 and locking barb 246 may be positioned in first interference coupling 216. Locking barb 246 is configured to be received within locking recess 222 to establish a releasable connection therebetween. In use, a clinician inserts at least a portion of elongated member 226 inside locking recess 222. During insertion of elongated member 226, locking barb 246 frictionally engages the inner surfaces defining locking recess 222, thereby establishing a releasable connection between first and second interference couplings 216, 220. Once first and second interference couplings 216, 220 are releasably connected to each other, coupling mechanism 214 provides fluid communication between first and second extension segments 208, 210. To establish fluid communication, first and second interference couplings 216, 220 jointly form a bore 244 extending therethrough. When coupling mechanism 214 secures first extension segment 208 to second extension segment 210, bore 244 fluidly connects first lumen 240 to second lumen 242.

As illustrated in FIGS. 5 and 6, some embodiments of medical system 200 further include an end cap 232 mountable adjacent to the first coupling 216 or any of the couplings or coupling mechanisms disclosed herein when second extension segment 210 is released from first interference coupling 216 or any other coupling described herein. End cap 232 is dimensioned to substantially close the first lumen 240 of first extension segment 208 to thereby prevent the passage of fluid. In one embodiment, end cap 232 includes an elongated member 234 with a locking barb 246 located at a distal end thereof. When end cap 232 is mounted on first interference coupling 216, locking barb 246 frictionally engages the inner surfaces defining locking recess 222. As a result, end cap 232 is releasably secured to the first interference coupling 216, as shown in FIGS. 3 and 4.

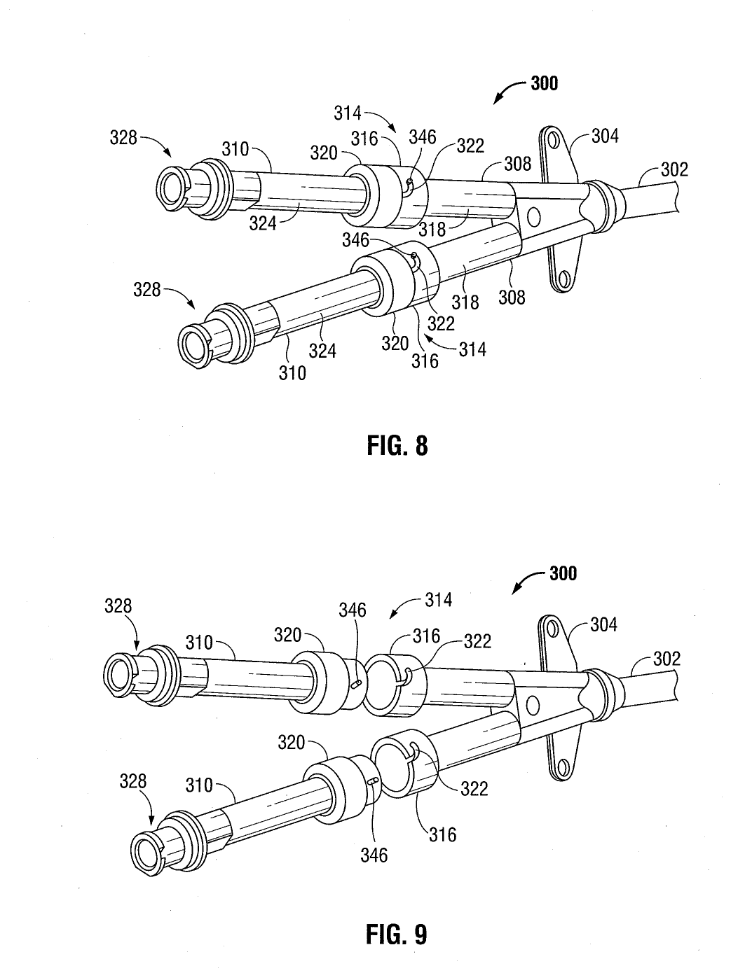

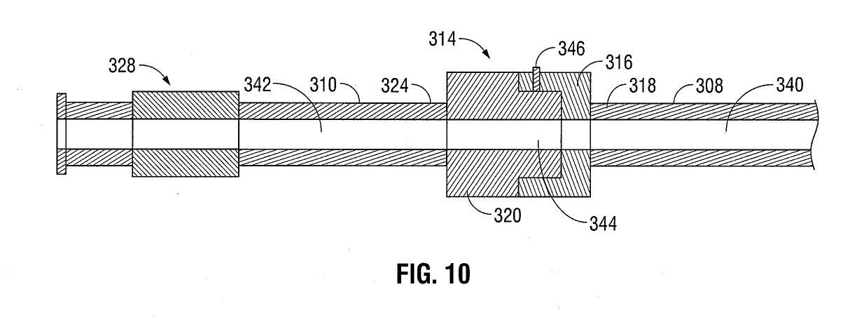

Referring to FIGS. 8-10, in an alternate embodiment, medical system 300 includes a coupling mechanism 314 adapted to connect first and second extension segments 308, 310 to establish fluid communication between first and second lumens 340, 342. Medical system 300 is substantially similar to medical system 100 except for coupling mechanism 314. Coupling mechanism 314 includes first and second bayonet couplings 316, 320, which collectively provide a releasable connection therebetween to allow selective connection and disconnection of first and second extension segments 308, 310. Specifically, first bayonet coupling 316 is attached to proximal end 318 of first extension segment 308 and second bayonet coupling 320 is secured to distal end 324 of second extension segment 310. First bayonet coupling 316 includes a slot 322 having a curved or hook-like profile. Second bayonet coupling 320 includes a pin 346 protruding radially therefrom. Slot 322 is adapted to slidably receive pin 346. To connect first bayonet coupling 316 to second bayonet coupling 320, a clinician positions pin 346 within slot 322 and rotates second bayonet coupling 320 until pin 346 reaches the closed end of slot 346. After interconnecting first and second bayonet couplings 316, 320, coupling mechanism 314 establishes fluid communication between first extension segment 308 and second extension segment 310. First and second bayonet couplings 316, 320 jointly define a bore 344 extending therethrough. Bore 344 fluidly connects first lumen 340 to second lumen 342 when first and second bayonet couplings 316, 320 are coupled to each other as illustrated in FIG. 10.

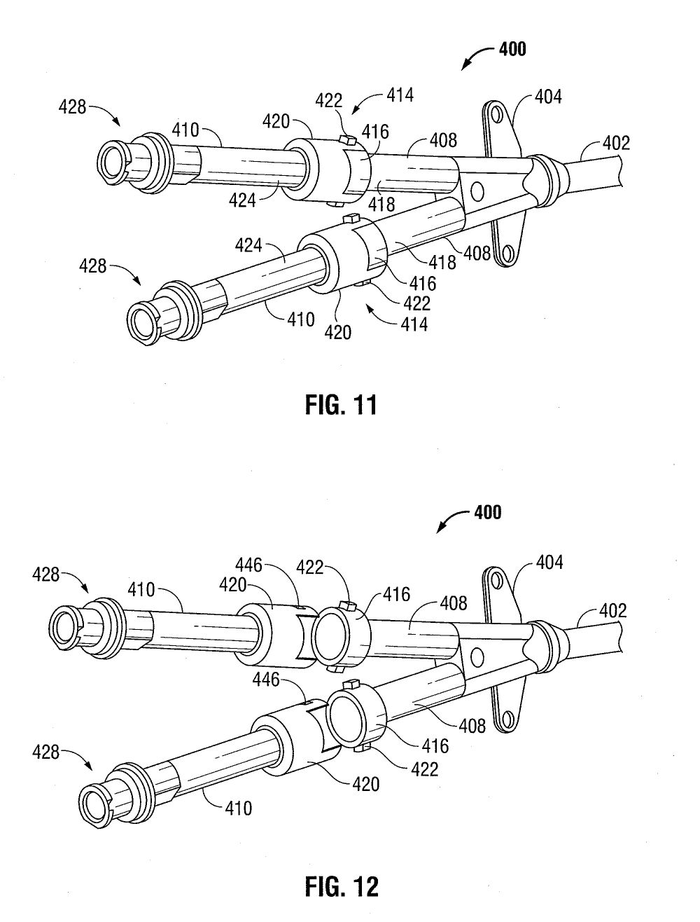

FIGS. 11-13 illustrate an alternative embodiment of medical system 100 which includes a coupling mechanism 414 adapted to connect the first and second extension segments 408, 410 to establish fluid communication between first and second lumens 440, 442. Medical system 400 is substantially similar to medical system 100 except for coupling mechanism 414. Coupling mechanism 414 includes a first snap fit coupling 416 and a second snap fit coupling 420. First and second snap fit couplings 416, 420 cooperate to provide a releasable connection therebetween to allow selective connection and disconnection of the first and second extension segments 408, 410. In particular, first snap fit coupling 416 is secured to the proximal end 418 of first extension segment 408 and second snap fit coupling 420 is attached to distal end 424 of second extension segment 410. First snap fit coupling 416 includes at least one locking lug 422 protruding radially therefrom. Second snap fit coupling 420 includes at least one locking extension 426 with an opening 446 formed thereon. Each opening 446 is adapted to securely receive locking lugs 422. In the embodiment depicted in FIGS. 11-13, first snap fit coupling 416 includes a pair of locking lugs 422 arranged in a diametrical opposed relation, and second snap fit coupling 420 has two locking extensions 416 that are also arranged in a diametrical opposed relation. In one embodiment, locking extensions 416 are biased toward each other.

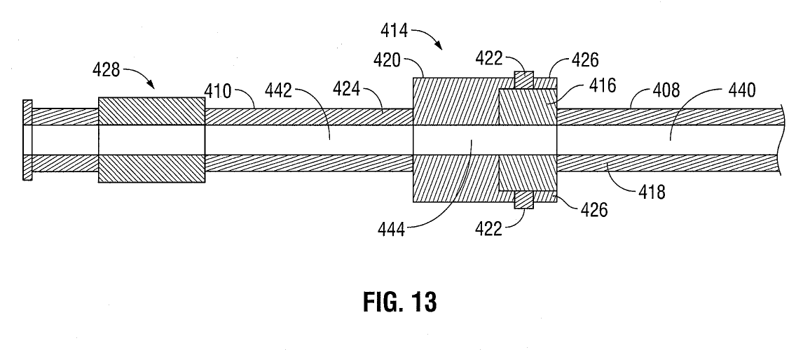

In use, a clinician connects first snap fit coupling 416 to second snap fit coupling 420 by placing locking extensions 426 over first snap fit coupling 416 so that openings 446 receive locking lugs 422, as shown in FIGS. 11 and 13. During this process, locking extensions 426 flex outwardly about locking lugs 422. When locking lugs 422 are positioned within openings 446, first and second snap fit couplings 416, 420 establish a releasable connection therebetween. This releasable connection between first and second snap fit couplings 416, 420 allows the clinician to selectively connect and disconnect first and second extension segments 408, 410. In addition, coupling mechanism 414 fluidly couples first lumen 440 to second lumen 442, when first and second snap fit couplings 416, 420 are connected to each other. To facilitate fluid communication between first and second lumens 440, 442, first and second snap fit couplings 416, 420 together form a bore 444 extending therethrough. Bore 444 establishes fluid communication between first and second lumens 440, 442 upon interconnection of first and second snap fit couplings 416, 420.

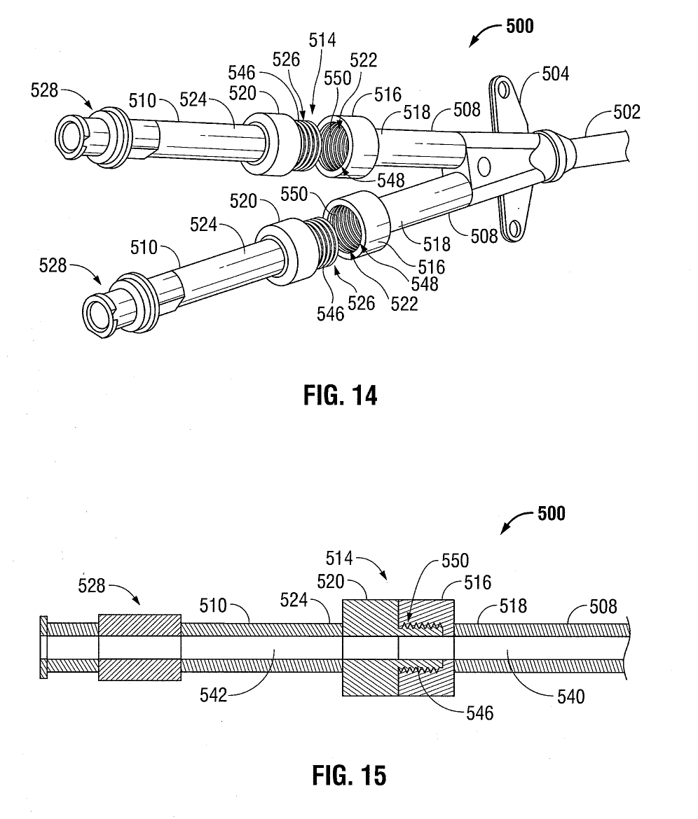

FIGS. 14 and 15 illustrate an alternative embodiment of medical system 500 which includes a coupling mechanism 514 adapted to connect first and second extension segments 508, 510 to establish fluid communication between first and second lumens 540, 542. Medical system 500 is substantially similar to medical system 100 except for coupling mechanism 514. Coupling mechanism 514 includes first and second threaded couplings 516, 520. First threaded coupling 516 is operatively connected to proximal end 518 of first extension segment 508, and second threaded coupling 520 is attached to distal end 524 of second extension segment 510. First and second threaded couplings 516, 520 cooperatively provide a releasable connection therebetween to permit selective connection and disconnection of first and second extension segments 508, 510. First threaded coupling 516 has an inner surface 548 defining a recess 522. Inner surface 548 includes an inner thread 550 formed thereabout. Recess 522 is dimensioned to receive an extension 526 of second threaded coupling 520. Extension 526 of second threaded coupling 520 has an external thread 546 formed thereabout which is configured to engage inner thread 550 when extension 526 is located within recess 522.

In operation, a clinician engages external thread 546 with inner thread 550 by initially inserting at least a portion of extension 526 inside recess 522. Then, the clinician rotates either first or second threaded coupling 516, 520 with respect to the other. As first or second threaded couplings 516, 520 rotate, external thread 546 engages inner thread 550 to releasably connect first threaded coupling 516 to second threaded coupling 520. Upon connection of first and second threaded couplings 516, 520, coupling mechanism 514 fluidly connects first lumen 540 to second lumen 542. To facilitate fluid communication between first and second lumens 540, 542, coupling mechanism 514 defines a bore 544 extending therethrough. As seen in FIG. 15, bore 544 establishes fluid communication between first and second lumens 540, 542 when first and second threaded couplings 516, 520 are connected to each other.

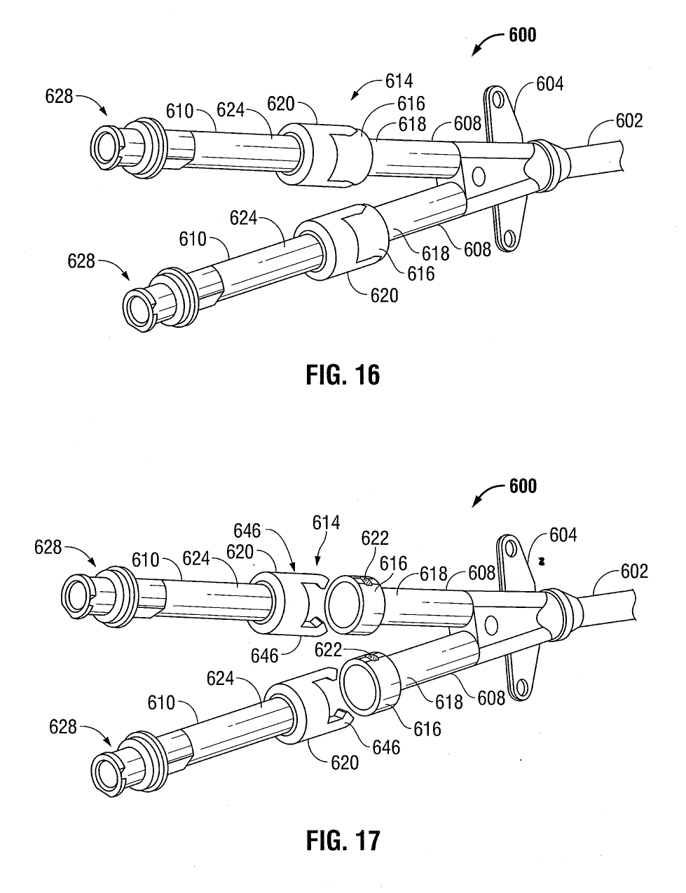

FIGS. 16-18 illustrate an alternative embodiment of medical system 600 which includes a coupling mechanism 614 adapted to connect the first and second extension segments 608, 610 to establish fluid communication between first and second lumens 640, 642. Medical system 600 is substantially similar to medical system 100 except for coupling mechanism 614. Coupling mechanism 614 includes first and second locking detent couplings 616, 620. First locking detent coupling 616 is attached to proximal end 618 of first extension segment 608, and second locking detent coupling 620 is secured to distal end 624 of second extension segment 610. First and second locking detent couplings 616, 620 cooperatively provide a releasable connection therebetween to permit selective connection and disconnection of first and second extension segments 608, 610. First locking detent coupling 616 has at least one notch 622 adapted to receive a locking detent 646 of second locking detent coupling 620. Locking detent 646 is pivotally or flexibly connected to second locking detent coupling 620 and is configured to securely engage notch 622 in a snap-fit type engagement. In one embodiment, first locking detent coupling 616 includes two notches 622, and second locking detent coupling 620 includes two locking detents 646. Coupling mechanism 614 defines a bore 644 adapted to fluidly connect first lumen 640 to second lumen 642 when first locking detent coupling 616 is connected to second locking detent coupling 620.

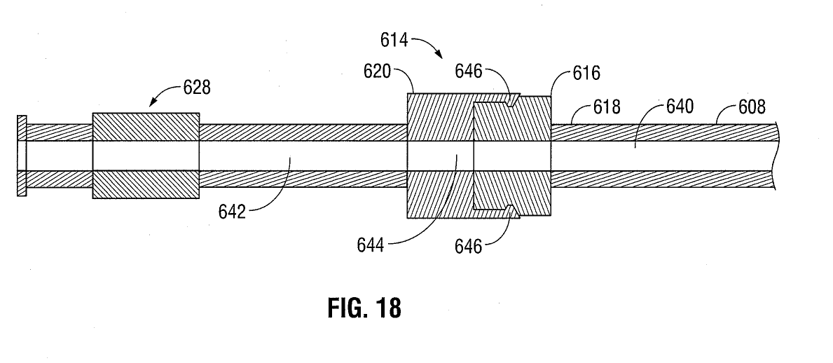

In operation, a clinician connects first extension segment 608 to second extension segment 610 by advancing second locking detent coupling 620 toward first locking detent coupling 616 until locking detents 646 fit into notches 622. During this process, locking detents 646 will flex outwardly as detents 646 engage first locking detent coupling 616 until detents 646 snap into notches 622. Once locking detents 646 are positioned within notches 622, bore 644 establishes fluid communication between first lumen 640 and second lumen 642, as shown in FIG. 18.

As discussed above, the presently disclosed medical systems (100, 200, 300, 400, 500, and 600) are particularly suited for hemodialysis procedures. Although the following medical procedure is discussed with respect to medical system 100, any of the presently disclosed medical systems can be employed in hemodialysis procedures. During hemodialysis procedures, blood travels between a hemodialysis machine and a subject's body through medical system 100. Typically, a clinician inserts medical system 100 into a subject's body to extract blood. Before extracting blood, the clinician releases second extension segment 110 and covers first coupling 116 with end cap 232 to prevent fluid from exiting through first extension segment 108. Once the clinician is prepared to commence the hemodialysis procedure, the clinician removes end cap 232 from first coupling 116 and utilizes coupling mechanism 116, or any other coupling mechanism disclosed herein, to fluidly connect second extension segment 110 to first extension segment 108. The clinician then fluidly connects the second extension segment 110 to the hemodialysis machine M through luer adapter 128. As soon as extension tube assemblies 106 establish fluid communication between hemodialysis machine M and medical system 100, the clinician can begin to extract blood from the subject. The extracted blood is then dialyzed and returned to the subject through medical system 100. After completing the hemodialysis procedure, the clinician disconnects second extension segment 110 from hemodialysis machine M and covers first coupling 116 with end cap 232 to prevent fluids from exiting first extension segment 108.

It will be understood that various modifications may be made to the embodiments of the presently disclosed medical systems. For instance, medical system 100 may include any other suitable coupling mechanism adapted to connect first extension segment 108 to second extension segment 110. Therefore, the above description should not be construed as limiting, but merely as exemplifications of embodiments. Those skilled in the art will envision other modifications within the scope and spirit of the present disclosure.

What is claimed is:

1. An extension tube assembly for fluidly connecting a catheter assembly to a hemodialysis machine, which comprises:
an extension tube member including first and second extension segments, the first extension segment adapted for connection to a catheter hub and defining a first lumen, the second extension segment adapted for fluid connection to a hemodialysis machine and defining a second lumen; and
a coupling mechanism adapted to connect the first and second extension segments to establish fluid communication between the first and second lumens, the coupling mechanism including:
a first coupling mounted on a distal end of the first extension segment, the first coupling including an elongate member extending distally therefrom, the elongate member having a barb disposed around a distal end thereof; and
a second coupling mounted on a proximal end of the second extension segment, the second coupling including a recess adapted to securely receive the elongate member to establish a secure connection between the first and second couplings;
wherein the first and second couplings cooperate to provide a releasable connection therebetween to permit selective connection and disconnection of the first and second extension segments; and
wherein the first and second couplings collectively form a bore when connected to each other, the bore being configured to establish fluid communication between the first and second lumens.

2. The extension tube assembly of claim 1, further comprising an end cap mountable on the first coupling of the first extension segment when the second coupling of the second extension segment is released from the first coupling of the first extension segment, the end cap being dimensioned to substantially close the first lumen of the first extension segment to prevent passage of fluid.

3. The extension tube assembly of claim 1, wherein the first extension segment includes a luer adapter mounted on a proximal end thereof.

4. An extension tube assembly for fluidly connecting a catheter assembly to a hemodialysis machine, which comprises:
an extension tube member including first and second extension segments, the first extension segment adapted for connection to a catheter hub and defining a first lumen, the second extension segment adapted for fluid connection to a hemodialysis machine and defining a second lumen; and
a coupling mechanism adapted to connect the first and second extension segments to establish fluid communication between the first and second lumens, the coupling mechanism including:
a first coupling mounted on a distal end of the first extension segment, the first coupling including an elongate member extending distally therefrom; and
a second coupling mounted on a proximal end of the second extension segment, the second coupling including a recess adapted to receive the elongate member;
wherein the first and second couplings jointly form a bayonet coupling mechanism to provide a releasable connection therebetween to permit selective connection and disconnection of the first and second extension segments, the coupling mechanism including:
a pin protruding radially from the elongate member; and
a slot formed on the second coupling, the slot having a curved profile.

5. The extension tube assembly of claim 4, wherein the second extension segment includes a luer adapter for fluid connection to the hemodialysis machine.

6. An extension tube assembly for fluidly connecting a catheter assembly to a hemodialysis machine, which comprises:
an extension tube member including first and second extension segments, the first extension segment adapted for connection to a catheter hub and defining a first lumen, the second extension segment adapted for fluid connection to a hemodialysis machine and defining a second lumen; and
a coupling mechanism adapted to connect the first and second extension segments to establish fluid communication between the first and second lumens, the coupling mechanism including:
a first coupling mounted on a distal end of the first extension segment, the first coupling including at least one flexible locking extension extending distally therefrom, the locking extension having an opening formed thereon; and
a second coupling mounted on a proximal end of the second extension segment, the second coupling including at least one lug extending radially therefrom, the at least one lug being adapted to be received in the opening of the at least one flexible locking extension to establish a secure connection between the first and second couplings;
wherein the first and second couplings cooperate to provide a releasable connection therebetween to permit selective connection and disconnection of the first and second extension segments; and
wherein the first and second couplings collectively form a bore when connected to each other, the bore being configured to establish fluid communication between the first and second lumens.

7. The extension tube assembly of claim 6, wherein the second extension segment includes a luer adapter for connection to the hemodialysis machine.

8. An extension tube assembly for fluidly connecting a catheter assembly to a hemodialysis machine, which comprises:
an extension tube member including first and second extension segments, the first extension segment adapted for connection to a catheter hub and defining a first lumen, the second extension segment adapted for fluid connection to a hemodialysis machine and defining a second lumen; and
a coupling mechanism adapted to connect the first and second extension segments to establish fluid communication between the first and second lumens, the coupling mechanism including:
a first coupling mounted on a distal end of the first extension segment, the first coupling including an extension extending distally therefrom, the extension having an external thread formed thereon; and
a second coupling mounted on a proximal end of the second extension segment, the second coupling including a recess, the recess having an internal thread formed therein, the internal thread being adapted to threadly engage the external thread to establish a secure connection between the first and second couplings;
wherein the first and second couplings cooperate to provide a releasable connection therebetween to permit selective connection and disconnection of the first and second extension segments; and
wherein the first and second couplings collectively form a bore when connected to each other, the bore being configured to establish fluid communication between the first and second lumens.

9. The extension tube assembly of claim 8, wherein the second extension segment includes a luer adapter for fluid connection to the hemodialysis machine.

10. An extension tube assembly for fluidly connecting a catheter assembly to a hemodialysis machine, which comprises:
an extension tube member including first and second extension segments, the first extension segment adapted for connection to a catheter hub and defining a first lumen, the second extension segment adapted for fluid connection to a hemodialysis machine and defining a second lumen; and
a coupling mechanism adapted to connect the first and second extension segments to establish fluid communication between the first and second lumens, the coupling mechanism including:
a first coupling mounted on a distal end of the first extension segment, the first coupling including a body and first and second arms pivotally secured to the body, each of the first and second arms having a locking detent at a distal end thereof; and
a second coupling mounted on a proximal end of the second extension segment, the second coupling including a first and second locking notches formed thereon, each notch being adapted to securely receive at least one of the locking detents to establish a secure connection between the first and second couplings;
wherein the first and second couplings cooperate to provide a releasable connection therebetween to permit selective connection and disconnection of the first and second extension segments; and
wherein the first and second couplings collectively form a bore when connected to each other, the bore being configured to establish fluid communication between the first and second lumens.

11. The extension tube assembly of claim 10, wherein the second extension segment includes a luer adapter for fluid connection to the hemodialysis machine.
